(19) United States
(12) Patent Application Publication
(10) Pub. No.: US 2003/0089548 A1
(43) Pub. Date: May 15, 2003

(54) In the ear hearing aid

(76) Inventors: Peter Frederiksen, Hellerup (DK); Jens T Balslev, Hellerup (DK)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 10/169,772

(22) PCT Filed: Jan. 19, 2001
(86) PCT No.: PCT/DK01/00043

(30) Foreign Application Priority Data
Jan. 19, 2000 (DK) PA 2000 00088

(51) Int. Cl. A61B 7/02

(52) U.S. Cl. 181/135

(57) ABSTRACT

The invention relates to a hearing aid for at least partly insertion into the car canal, the hearing aid comprising a housing, a faceplate, a battery drawer, a microphone having directional characteristics, where the microphone comprises a microphone housing having a number of surfaces each surrounded by at least three edges, each edge forming a boundary towards another surface, where two inlet openings are arranged with a predetermined distance in the same surface of the microphone housing, where the microphone is mounted at least partly in the faceplate, where the inlet openings for the sound to the directional microphone are located embedded in the faceplate. The invention further relates to a faceplate and a sealing element for sealing between a faceplate and a transducer.

### FIELD OF THE INVENTION

The invention relates to so-called in the ear (ITE) hearing aids, which are characterized by that at least part of the hearing aid is inserted in the ear canal of the bearing aid user during use. More specifically the invention relates to ITE hearing aids, which includes directional microphone characteristics.

### BACKGROUND OF THE INVENTION

ITE hearing aids are desirable for many hearing aid users due to the size of these, which allow the hearing aid to remain at least partly hidden and hence less appearing to others. Over the years a number of different types of microphones have been manufactured. These count omni directional microphones and directional microphones. Many hearing aid users do in different situations experience a preference for a directional microphone, whilst in other situations an omni directional microphone is preferred. In order to satisfy the need for choosing a specific microphone characteristic depending on the environment a number of different solutions have been suggested for ITE hearing aids comprising a directional microphone and an omni directional microphone or a combined unit comprising both these microphone characteristics. A common problem for these previously known ITE hearing aids having both the directional and the omni directional characteristics have been the size of these hearing aids, which has grown due to the implementation of the extra microphone or the unit as mentioned above. This is of course a problem as the ITE hearing aid often is desired due the possibility of at least partly hiding this. Making the ITE hearing aid bigger will make this less desirable from an appearance point of view. One objective of the present invention is to suggest a construction concept for an ITE hearing aid, which features the desired directional and the omni direction HI microphone characteristics and which at the same time makes it possible to achieve a small size of the hearing aid. A further objective is to provide a faceplate solution for a ITE hearing aid, which will make it possible to reach a small size of the hearing aid. A still further objective of the present invention is to provide a sealing solution for a transducer in a hearing aid, which will make it possible to provide a hearing aid solution with an even smaller size.

### SUMMARY OF THE INVENTION

According to the invention the first objective is achieved by means of the hearing aid as defined in claim 1.

By means of such construction the hearing aid may be manufactured to be more compact and hence more cosmetically attractive. Omitting the inlet tubes, which usually are present on previously known microphones means that the microphone may be embedded deeper into the faceplate than possible by previously known microphones.

To maintain a proper sealing effect a sealing element is provided. According to the invention the faceplate aspect of the invention is achieved by means of a faceplate as defined in claim 5. By means of such faceplate a more cosmetically attractive hearing aid may be manufactured.

According to the invention the sealing aspect of the invention is achieved by means of a sealing element as defined in claim 8 and 10. By means of such sealing element the sealing between faceplate and microphone may be achieved in a more efficient and more reliable manner especially when the microphone does not show the previously used inlet tubes.

The invention will be described more detailed in the following description of preferred embodiments, showing different views of faceplates, which are used for making a ITE hearing instrument.

### BRIEF DESCRIPTION OF THE DRAWINGS

FIG. 1 is a isometric view of a faceplate assembly;

FIG. 2 is a top view of the faceplate of FIG. 1;

FIG. 3 is a bottom view of the faceplate of FIG. 1;

FIG. 4 is a sectional view after the line A-A in FIG. 3;

FIG. 5 is a sectional view after the line B-B in FIG. 3.

### DESCRIPTION OF THE PREFERRED EMBODIMENT

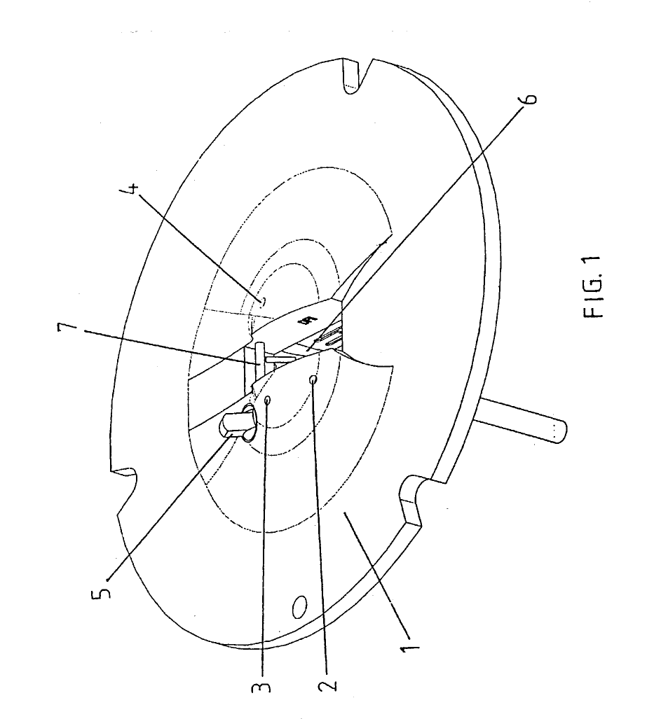

From FIG. 1 a faceplate 1 appears, which forms part of a hearing aid according to the invention. A faceplate is a well-known construction part within the hearing aid industry. The faceplate forms together with a customized shell (shaped according to an impression of the hearing aid user's ear) the housing of the ITE hearing aid. The faceplate 1 comprises with a compartment 6 for housing a battery drawer (not shown). The battery drawer is hinged pivotally on the shaft 7. Next to the battery drawer are at one side two inlet openings 2,3 for sound inlet to a directional microphone having two inlet openings. On the opposite side of the compartment 6 a single opening is provided for sound inlet to a microphone having a single inlet opening. At the same side of the 4 compartment 6 as the two openings 2,3 a switch 5 is provided for providing a possibility of shifting between the different microphones and hence changing the directional characteristics of the hearing aid which is manufactured from the faceplate and a shell and the further necessary components thereof. It Is obvious that tile hearing aid will comprise an amplifier and output means. The faceplate is mounted on a shell, which together with the faceplate form a housing. These parts are not shown here but would be obvious components to assemble for the skilled person.

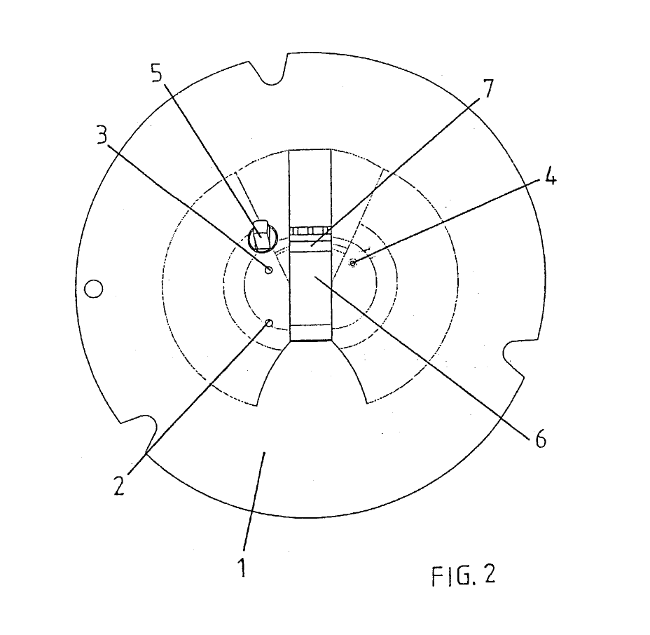

From FIG. 2 the faceplate appears seen from above.

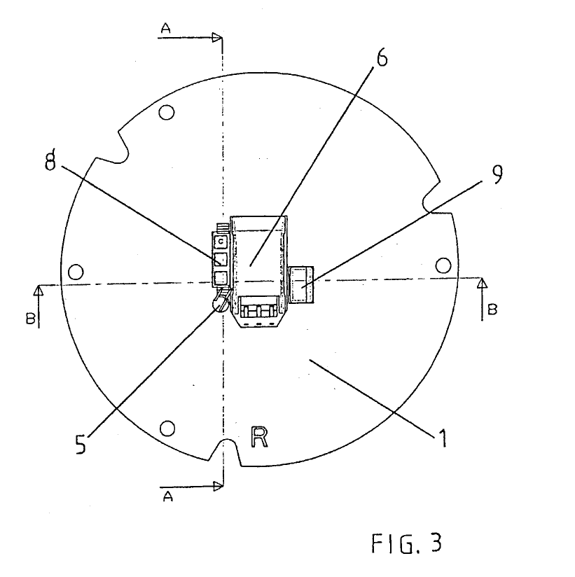

From FIG. 3 the faceplate appears seen from the bottom side. The two microphones appear in this view. The microphone 8 is a directional microphone having two inlet openings. The microphone 9 is an omni-directional microphone having a single inlet opening.

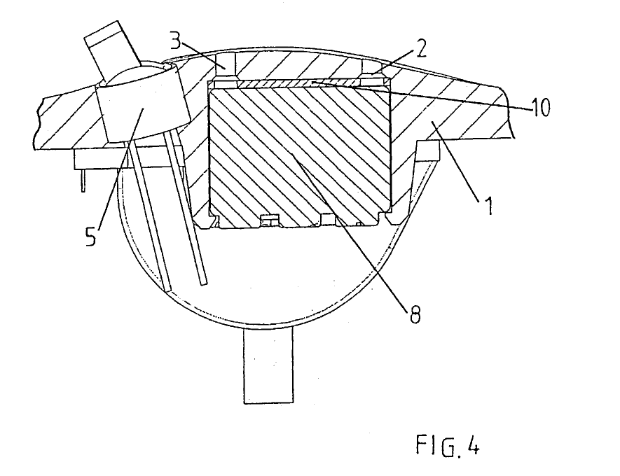

From FIG. 4 the faceplate 1 appears in a sectional view and the directional microphone 8. The directional microphone 8 has two inlet openings in a planar surface. Omitting the usual inlet tubes in the microphone allows for embedding the microphone deeper into the faceplate and hence achieves an even smaller hearing aid. The inlet openings in the microphone are in line with the openings 2,3 in the faceplate, although it is possible to offset the openings slightly, e.g. a distance corresponding to the radius of the inlet opening. The microphone is sealed against the faceplate by means of a sealing element 10 having two openings in line with the openings in the faceplate and the microphone. The microphone is held in place by means of locking arms.

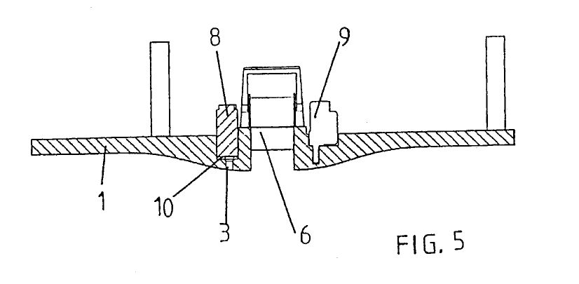

From FIG. 5 appears in 3 further sectional view the location of the microphones 8,9 in relation to the compartment 6 for the battery drawer. It appears that the microphones are placed in a close vicinity of this compartment In order to achieve the most compact construction and hence the cosmetically most attractive hearing aid.

A possibility of having a single opening in the faceplate, where the acoustic inlets in the microphone face the environment through that opening also exists. Hereby the single opening is preferably covered by a filter unit.

1. A hearing aid for at least partly insertion into the ear canal. the hearing aid comprising: a hearing aid housing, a faceplate, a microphone having directional characteristics, where the microphone comprises a microphone housing having a number of surfaces, where two inlet openings are arranged with a predetermined distance in one inlet surface of the microphone housing, where the microphone is embedded at least partly in the faceplate, where a sealing element is provided in such a manner that the signals will not propagate between the microphone housing and the faceplate towards the inner of the hearing aid housing.

2. A hearing aid according to claim 1, further comprising at least one sealing element, which is placed between the microphone and the faceplate for sealing the area around the microphone inlets towards the faceplate.

3. A hearing aid according to claim 2, where the sealing element comprises a substantially planar base element with at least one through hole.

4. A hearing aid according to claim 3, where a bead is formed on the sealing element the bead surrounding a microphone inlet.

5. A faceplate for use in a hearing aid for at least partly insertion in an ear canal, the faceplate comprising: two inlet openings for the sound to a directional microphone having two inlet ports are located with essentially the same distance as the inlet openings in the microphone housing, where a cavity is provided for at least partly embedding a microphone in the faceplate.

6. A faceplate according to claim 5, where two flexible arms are provided for holding a microphone embedded in the faceplate.

7. A sealing element for sealing a microphone having at least one opening in a surface thereof, towards a faceplate of an ITE hearing aid, where the sealing element comprises a substantially planar base element with at least one through hole.

8. A sealing element according to claim 7, where a bead is formed on the sealing element the bead surrounding a microphone Inlet.

9. A sealing element for sealing a microphone having at least one opening in a Surface thereof towards a faceplate of an ITE hearing aid where the sealing element comprises a structure corresponding to the outer circumference of the surface in which the at least one opening is located.
